(19) United States
(12) Patent Application Publication
(10) Pub. No.: US 2014/0179987 A1
(43) Pub. Date: Jun. 26, 2014

(54) Kit for Placing Fixation Devices

(71) Applicant: Boston Scientific Scimed, Inc., Maple Grove, MN (US)

(72) Inventor: Dennis Miller, Whitefish Bay, WI (US)

(73) Assignee: Boston Scientific Scimed, Inc., Maple Grove, MN (US)

(21) Appl. No.: 14/063,770

(22) Filed: Oct. 25, 2013

Related U.S. Application Data
(63) Continuation-in-part of application No. 13/072,333, filed on Mar. 25, 2011, now abandoned.

(51) Int. Cl. A61F 2/00

(52) U.S. Cl. CPC A61F 2/0022
USPC 600/30

(57) ABSTRACT

A fixation device can be delivered to the body of a patient by coupling the fixation device to a delivery instrument at a head of the fixation device. Engaging the fixation device to the delivery instrument at the fixation device's head allows the fixation device to be secured to a tissue and/or ligament of the patient. The fixation device can be a sling for placement around the bladder neck of a female patient.

### FIELD OF THE INVENTION

The present invention generally is directed to a kit for the placement of a fixation device within the body of a patient.

### BACKGROUND OF THE INVENTION

Conditions such as rectocele, cystocele, enterocele, vaginal prolapse, and protocele involve tissues or organs that have been damaged, prolapsed, weakened, or otherwise herniated. A prolapse refers to the slipping of an organ, or organ part, from its normal position. For example, a prolapse of the rectum refers to the protrusion of the rectum through the anus. Rectocele is the prolapse of the rectum into the perineum. A prolapse of the uterus refers to the falling of the uterus into the vagina due to stretching and laxity of its supporting structures. Vaginal vault prolapse refers to the prolapse of the cephalad extreme of the vaginal wall toward, through, and beyond the introitus. Cystocele (i.e., vesicocele) is a hernia formed by the downward and backward displacement of the urinary bladder toward the vaginal orifice, due most commonly to weakening of the musculature during childbirth. However, any abnormal descent of the anterior vaginal wall and bladder base at rest or with strain is considered cystocele. Enterocele is a hernia of the intestine, though the term is also used to refer specifically to herniation of the pelvic peritoneum through the rectouterine pouch (i.e., posterior vaginal, rectovaginal, cul-de-sac, or Douglas' pouch hernia). Proctocele is a prolapse of the mucous coat of the rectum due mostly from relaxation of the sphincter. Treatment of these conditions frequently requires a sling, such as a mesh sling, implanted at the anatomical site-requiring repair.

Stress urinary incontinence (SUI) primarily affects women and generally is caused by two conditions that may occur independently or in combination, namely, intrinsic sphincter deficiency (ISD) and hypermobility. In ISD, the urinary sphincter valve, located within the urethra, fails to close properly, causing urine to leak out of the urethra during stressful actions. Hypermobility is a condition in which the pelvic floor is distended, weakened, or damaged, causing the bladder neck and proximal urethra to rotate and descend in response to increases in intra-abdominal pressure (e.g., due to sneezing, coughing, straining, etc.), resulting in insufficient response time to promote urethral closure and, consequently, in urine leakage and/or flow.

Biological factors that may affect hypermobility include: poor endopelvic fascia muscle tone (from, for example, age or limited activity), endopelvic fascia muscle stretch/tear from trauma (e.g., childbirth), endopelvic fascia/arcus tendenious (muscle/ligament) separation (lateral defect), hormone (e.g., estrogen) deficiency, concombinant defects (e.g., cystocele, enterocele, and ureteral prolapse), and vaginal prolapse. Traditional treatment methods include urethra or bladder neck stabilization slings in which a sling is placed under the mid-urethra or bladder neck to provide a platform preventing over distention.

Slings are traditionally placed under the urethra or bladder neck to provide a urethral platform limiting endopelvic fascia drop while providing compression to the urethral sphincter to improve coaptation. The urethral placement location provides mechanical stability to a less moveable anatomical structure. Bladder neck slings traditionally have been affixed in the desired location using a bone anchoring method. Mid-urethral slings, being placed in a low mobility area, may be placed using an anchorless approach. Recognizing that minimal tension, if any, is necessary, a physician may need only to secure a mid-urethra sling through the endopelvic fascia. The sling in this placement provides a fulcrum about which the pelvic floor will drop (taking advantage of the hypermobility condition of the patient) and a urethral “kink” or higher resistance to obstruct urine flow during high stress conditions.

A known method for stabilizing organs and tissues within the pelvic region involves the use of bone anchors. Deployment of a bone anchor requires drilling a hole in a bone, either by using a separate drilling instrument or by utilizing the anchor itself as a drilling tool. Bone anchors generally have one or more barbs that project outward to prevent the anchor from exiting the hole. Such anchors generally are not amenable to implantation in soft tissues, since the barbs would tear the soft tissue, causing irritation and/or passage of the anchor back through the tissue.

Other known methods include making one or more incisions in a patient's abdomen. For example, one method for treating female stress urinary incontinence involves supporting the urethra with an implant anchored in the patient's skin after the implant has been passed through the skin of the patient's abdomen.

### SUMMARY OF THE INVENTION

Illustrative embodiments according to the invention are directed towards securing a fixation device to a treatment area within the body of a patient such as a human or other mammal. Some embodiments are directed towards a fixation device sized to be coupled to a delivery instrument for delivering the fixation device to a treatment area within the body by coupling the fixation device to the delivery instrument at a fixation device head of the instrument. Coupling the fixation device to the head of the delivery instrument allows the fixation device to be delivered to a tissue and/or ligament within the body and subsequently secured to that tissue and/or ligament.

In one aspect, the invention relates to an instrument for delivering a fixation device to a tissue comprises a distal portion including a carrier capable of extending from a retracted position to an extended position upon actuation by a user. The carrier comprises a side slot which allows the carrier to receive a fixation device. A shaft of the fixation device passes through the slot of the carrier, and a fixation device head is positioned at a distal end of the carrier. The instrument also comprises a catch for securing the fixation device head once the fixation device head has been driven through the tissue as the carrier moves from the retracted position to the extended position.

In another aspect, the invention involves a fixation device comprises a head engaged to a shaft. The head and the shaft are sized to be received by an extendable carrier of a delivery instrument, and the head is sized to be secured within a catch of the delivery instrument. A sling can be engaged to the shaft of the fixation device.

In yet another aspect, the invention features a sling which comprises a first fixation device engaged to a first end of the sling. The first fixation device can comprise a head of the first fixation device engaged to a shaft. The head and the shaft are sized to be received by an extendable carrier of a delivery instrument, and the head is sized to be secured within a catch of the delivery instrument. A plurality of the fixation devices can be engaged to the sling.

Numerous other aspects, features, and advantages of the present invention will be made apparent from the following detailed description together with the drawings figures.

### BRIEF DESCRIPTION OF THE DRAWINGS

The disclosed embodiments will be further explained with reference to the attached drawings, wherein like structures are referred to by like numerals throughout the several views. The drawings are not necessarily to scale, the emphasis having instead been generally placed upon illustrating the principles of the invention and the disclosed embodiments.

FIG. 1A is a schematic plan view of an embodiment of a delivery instrument engaged to an embodiment of a presently disclosed fixation device;

FIGS. 1B and 1C (fixation device not attached) are schematic cross-sectional views of a proximal portion and a distal portion of the delivery instrument of FIG. 1A;

FIG. 2 shows an embodiment of a fixation device engaged to a delivery instrument;

FIG. 3 shows an embodiment wherein the fixation device has passed through a tissue, a fixation device head has been engaged in a catch of a delivery instrument, and the fixation device is being pulled through a tissue and/or a ligament by withdrawing the delivery instrument;

FIG. 4 shows an embodiment of a fixation device wherein the fixation device comprises a plurality of barbs;

FIG. 5 shows an embodiment wherein a plurality of fixation devices are engaged to a graft;

FIG. 6 shows another embodiment wherein a plurality of fixation devices are engaged to a graft;

FIG. 7 shows an embodiment of a fixation device comprising a T-shaped protrusion wherein the fixation device is not engaged to a sling material;

FIG. 8 shows an embodiment of a fixation device and a lock button wherein the lock button engages a protrusion on the fixation device;

FIG. 9A is a schematic plan view of a fixation device head coupled to a shaft for use in a delivery instrument in accordance with the invention; and

FIG. 9B is a schematic perspective view of a catch for use with the delivery instrument of FIG. 1A.

### DETAILED DESCRIPTION OF THE INVENTION

Illustrative embodiments according to the invention are directed towards a fixation device and an instrument and method for securing the fixation device to a treatment area. More specifically, particular illustrative embodiments described herein are directed towards a fixation device sized to be coupled to a delivery instrument, and a method of delivering the fixation device to a treatment area by coupling the fixation device to the delivery instrument at a fixation device head. Coupling the fixation device to the delivery instrument at the fixation device head allows the fixation device to be delivered and secured to a tissue and/or ligament.

In one embodiment, a method for the placement of a sling material involves engaging a first fixation device to a first end of a sling material and engaging a second fixation device to a second end of the sling material. The fixation device head of the first fixation device is engaged to an extendable carrier of the delivery instrument. Next, the fixation device head of the first fixation device is pushed through a tissue and/or ligament and the fixation device head is secured in a catch of the delivery instrument. Once the head of the fixation device is secured in the catch, the delivery instrument is withdrawn from the tissue a desired distance in order to further secure the fixation device to the tissue at a desired tension. The head of the first fixation device head then is disengaged from the delivery instrument.

Once the first fixation device is secured in the tissue, the head of the second fixation device is engaged to the extendable carrier of the delivery instrument. As such, the second fixation device may be delivered to a second desired location in the same manner as described above. A plurality of fixation devices can be engaged to a sling material, and each fixation device is thereby delivered to the treatment area. Any number of fixation devices engaged to a sling material (or any other material) are within the scope of the present invention.

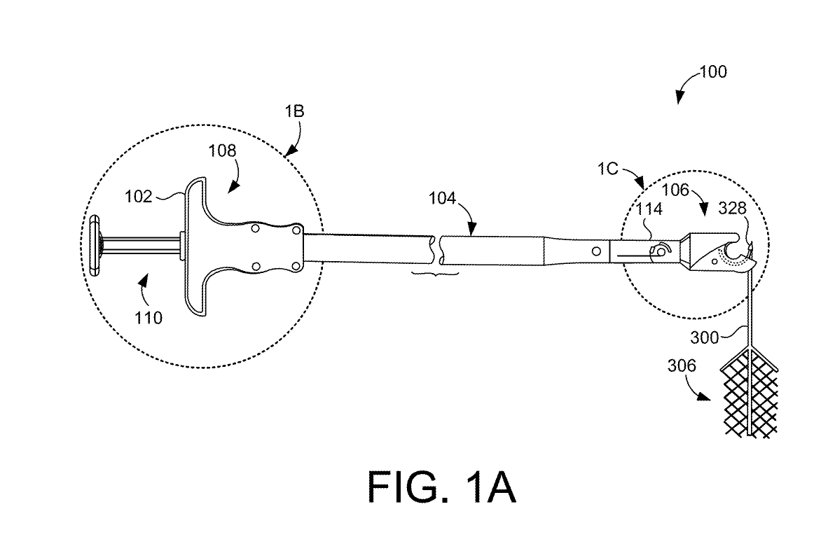

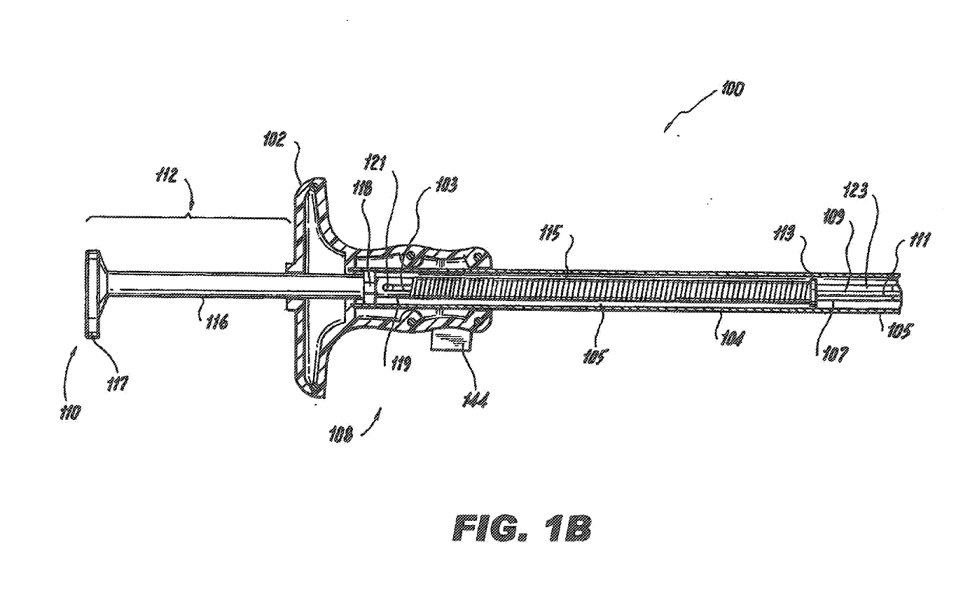

An embodiment of a delivery instrument 100 is shown in FIGS. 1A-1C. FIG. 1A depicts a delivery instrument 100 including a handle 102, an elongate body member 104, and a fixation head deployment mechanism 110. The delivery instrument 100 also includes a distal portion 106 and a proximal portion 108. The elongate body member 104 is mechanically coupled to the handle 102 at the proximal portion 108 and the delivery components are at least partially disposed within the distal portion 106 of the delivery instrument 100.

As shown in FIG. 1A, a fixation device 306 is engaged to the distal portion 106 of the delivery instrument 100 by placing the fixation device head 328 in an extendable carrier (shown in FIG. 1C). The carrier 124 comprises a side slot. When coupling the fixation device 306 to the carrier 124, the shaft 300 of the fixation device 306 is sized to pass through the side slot and the fixation device head 328 is positioned on top of an opening at a distal end of the carrier. The diameter of the fixation device head 328 is larger than the diameter of a lumen of the carrier. As such, the fixation device head 328 is positioned over the distal opening of the carrier while a portion of the shaft 300 of the fixation device resides within the lumen of the carrier. As such, when the fixation head deployment mechanism 110 is depressed, the carrier 124 drives the fixation device head 328 through a tissue until the fixation device head 328 is secured in a catch. Once secured in a catch, the carrier retracts to its original position and thus disengages from the fixation device head 328. Once the fixation device head 328 is secured in the catch, the delivery instrument 100 may be withdrawn a distance to pull the fixation device 306 a desired distance through the tissue. Finally, the fixation device head 328 is disengaged from the carrier once the fixation device has been successfully placed in a tissue. An embodiment of these components will be shown in detail in FIGS. 1B and 1C.

The handle 102 of the delivery instrument 100 could take a variety of forms, for example, the handle 102 could be one of the types used with Boston Scientific Corporation suturing systems, in particular the Capio® Push & Catch suturing system. Generally, the fixation device deployment mechanism 110 extends longitudinally through the elongate body member 104 to the distal portion 106 of the delivery instrument 100, where the fixation device head deployment mechanism 110 is coupled to a carrier 124 (FIG. 1C). The fixation device head deployment mechanism 110 moves the carrier 124 between a retracted position and a extended position. The fixation device deployment mechanism 110 is shown in greater detail in FIGS. 1B and 1C.

Referring to FIG. 1B, the proximal portion 108 of the delivery instrument 100 includes the handle 102, the elongate body member 104, and the fixation device deployment mechanism 110. The fixation device deployment mechanism 110 includes an actuator 112 (button 117, shaft 116), a bearing 118, a button end 119, and a hole 121. The bearing 118 rides along a cylindrical surface 105 that is formed by the inside diameter of the elongate body member 104. A wireform 103 is inserted into the hole 121, coupling it to the actuator button 117. A spring 115 encircles the wireform 103, abuts the button end 119, and is compressed between the button end 119 and a spring washer 113. The spring washer 113 is seated upon a center tube 107. The center tube 107 is housed by the cylindrical surface 105 and is constrained in the distal portion 106. A pusher wire 111 is attached to the wireform 103 by means of a weld, a coupling, adhesive or other means, and is slidably disposed within a guidance sleeve 109, the sleeve 109 being disposed within a cylindrical surface 123 formed by the inside diameter of the center tube 107. In an embodiment, the pusher wire 111 is constructed of nitinol, so chosen for its combination of properties that allow for bendability and high column strength when constrained. Nitinol is a nickel-titanium alloy. Those skilled in the art will recognize that a wire made of various materials are within the spirit and scope of the present invention.

Referring to FIG. 1C, the distal portion 106 of the delivery instrument 100 of FIG. 1A includes the elongate body member 104, the fixation device deployment mechanism 110, an articulation mechanism 114, a curved portion 126, and a catch 122. Referring again to the fixation device deployment mechanism 110, the pusher wire 111 is attached by welding or other means to a coupling 150, which is slidably disposed within a track 152. The coupling 150 is attached to a carrier wire 154, which by virtue of its attachment to the coupling 150 is also slidably disposed within the track 152. The carrier wire 154 is mechanically coupled to an extendable carrier 124 by means of a weld, a coupling, adhesives, or other means. The coupling 150 abuts a backstop washer 156 that is slidably disposed about the pusher wire 111 and is contained within a pocket 160 that includes a back wall 162, against which the backstop washer 156 rests. The track 152 terminates distally in a pocket 164 that includes a wall 166. A downstop washer 158 is slidably disposed about the carrier wire 154 and constrained within the pocket 164.

In some embodiments, the delivery instrument 100 may include the articulation mechanism 114. The articulation mechanism 114 is disposed in the elongate body member 104 proximate the distal portion 106 (FIG. 1C). The articulation mechanism 114 facilitates the rotation (in the directions indicated by arrow 182) and positioning of the distal end 106 of the delivery instrument 100. In addition, the elongate body 104 can be substantially linear or may include one or more bends. The articulation mechanism 114 and/or bend(s) can facilitate access to deep and/or difficult to reach areas within the patient.

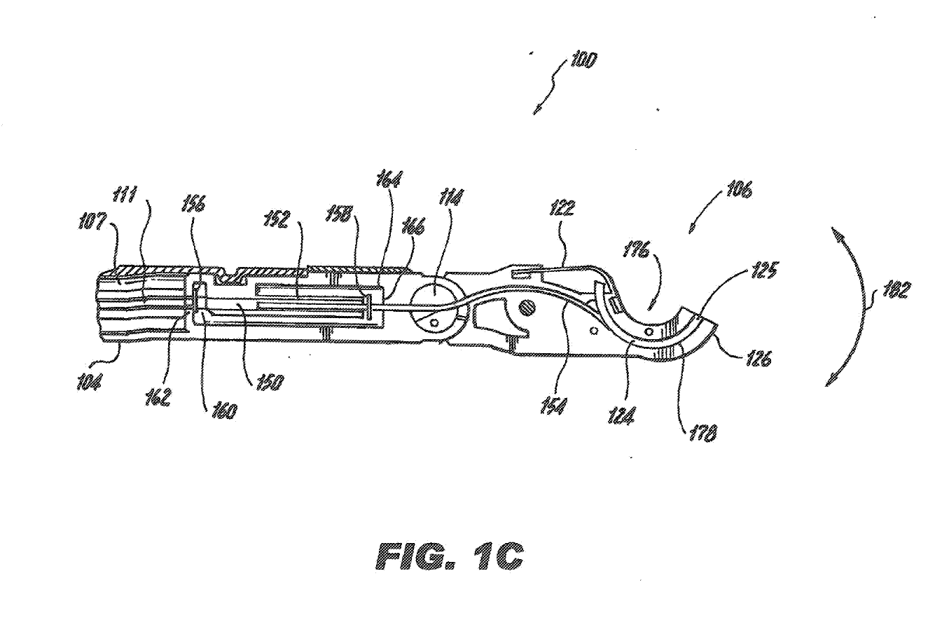

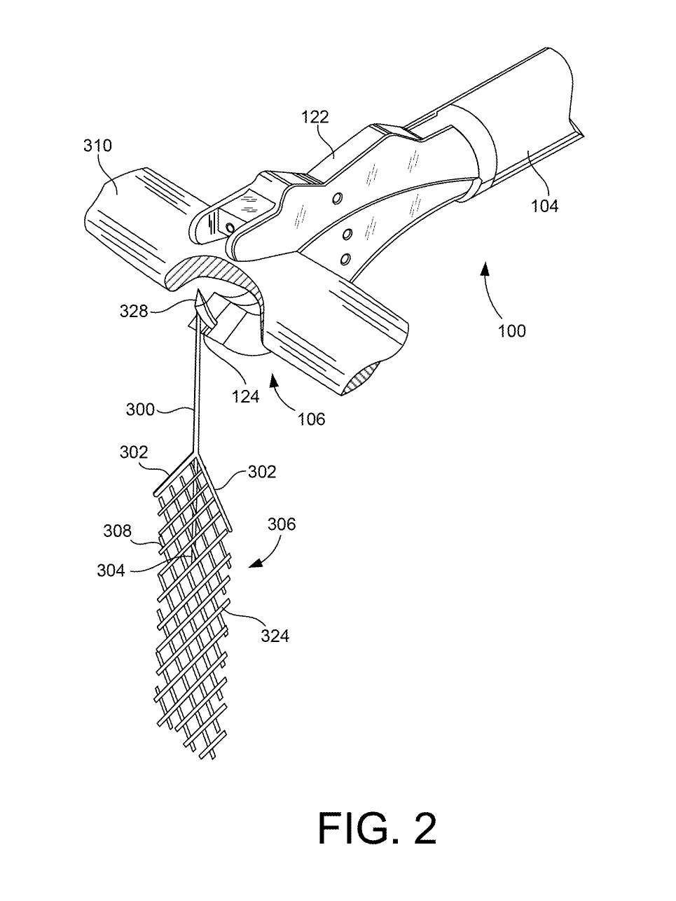

FIG. 1C shows a distal opening 125 of the carrier 124. As discussed above, the carrier comprises a side slot which begins at the distal opening 125. In addition, the curved section 126 of the distal end comprises a slot 127 (shown in FIG. 3) which aligns with the side slot of the carrier 124. Aligning the slot of the carrier with the slot of the curved section 126 allows the shaft 300 of the fixation device to reside within a portion of the carrier while the fixation device head 328 rests on top of the distal opening 125 of the carrier (as shown in FIG. 1A and FIG. 2).

FIG. 2 shows a presently disclosed embodiment of a delivery instrument 100 engaged to a fixation device 306. The fixation device 306 is engaged to the delivery instrument 100 by inserting the fixation device head 328 into an extendable carrier 124. As described above in relation to FIGS. 1A-1C, the extendable carrier 124 may be in a retracted position or an extended position. As shown in FIG. 2, the extendable carrier 124 is in a retracted position.

As shown in FIG. 2, an embodiment of the fixation device 306 comprises a shaft 300 which runs from the fixation device head 328 and ends with the beginning of a sling material 324. In an embodiment, the sling material 324 is a mesh. In an embodiment, the sling material 324 is a graft. In an embodiment, the sling material 324 may comprise a drug. Those skilled in the art will recognize that various other materials are within the spirit and scope of the present invention.

When the fixation device head 328 is coupled to the delivery instrument 100, the shoulder of the fixation device head 328 rests on the front edge of the extendable carrier 124, while the flexible shaft 300 of the fixation device 306 slides into the extendable carrier 124 through a slot and exits by a side port. The length of the flexible shaft 306 can vary depending on the need to exit the body for reloading for securing an additional fixation device 306.

In the embodiment shown in FIG. 2, a first and a second protrusion 302 extend from the flexible shaft 300 to encapsulate the sling material (i.e., a mesh or graft) and act as a leading edge. In an embodiment, the leading edge is stiffer than the sling material and angled to provide support to keep the sling material width extended yet collapsible to follow the flexible shaft 300 through a ligament and/or tissue 310. The ends of the leading edge 302 can also act as a barb for anchoring the fixation device 306 in place.

In an embodiment, the flexible shaft 300 extends through the sling material and act as a backbone or support segment 304. The backbone segment 304 provides strength to the sling material 324 to prevent the sling material 324 from unraveling as the fixation device 306 is pulled through a tissue and/or ligament 310.

In an embodiment, the sling material 324 comprises a plurality of tangs 308. The tangs 308 engage the tissue and/or ligament 310 in order to help secure the fixation device 306 to the tissue and/or ligament 310.

In an embodiment, the fixation device 306 comprises a medical grade, implantable polypropylene. In an embodiment, the fixation device 306 comprises a bio-absorbable material. Those skilled in the art will recognize that various materials are within the spirit and scope of the present invention.

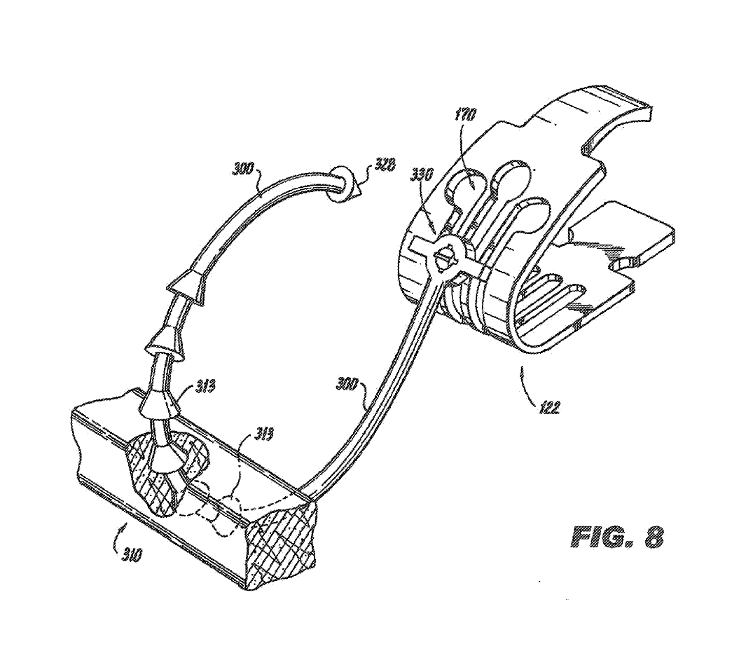

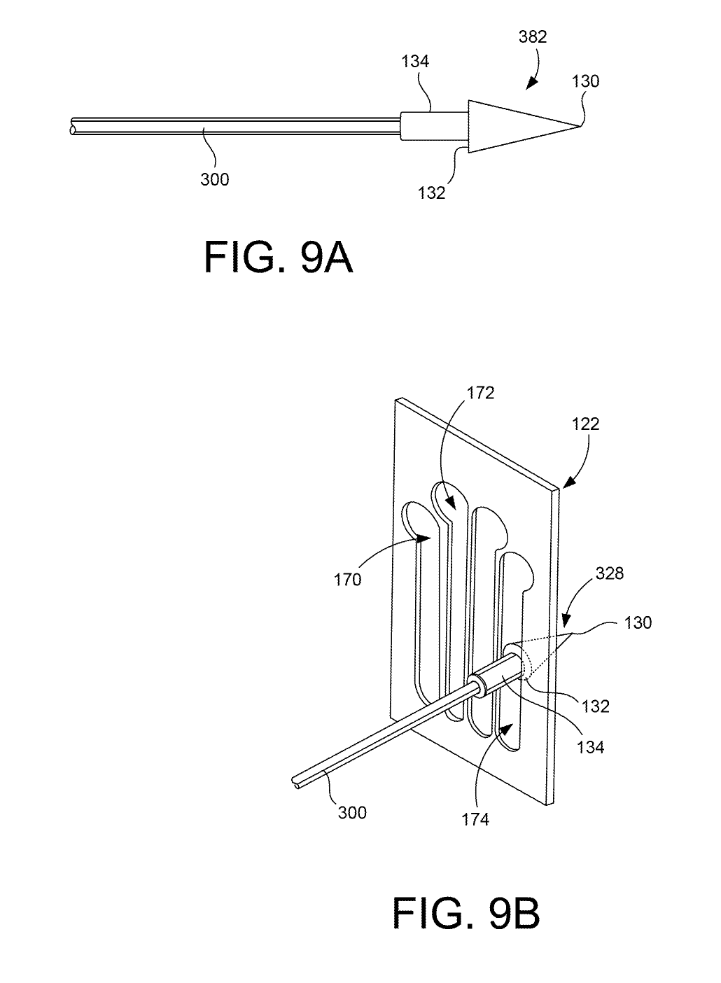

FIG. 2 shows an embodiment wherein the delivery instrument 100 comprises the extendable carrier 124 in a retracted orientation. By engaging the fixation device deployment mechanism 110 (discussed above in relation to FIGS. 1A-1C), the extendable carrier 124 is extended full to allow the fixation device head 328 to pierce ligament or tissue and be received in the catch 122. As will be shown below, the pointed fixation device head 328 spreads the slot of the catch 122 as it passes into the catch 122. The slot then narrows as the extendable carrier 124 retracts, trapping the head by its wider shoulder within the catch 122 (see FIGS. 9A and 9B).

FIG. 3 shows an embodiment of the presently disclosed delivery instrument 100 wherein the fixation device head 328 has passed through a tissue 310 and is now secured in the catch 122. Once the fixation device head 328 is retained in the catch of the delivery instrument 100, the delivery instrument 100 is pulled away from the treatment area in the general direction of arrow “A”. As the delivery instrument 100 is withdrawn from the tissue and/or ligament 310, the fixation device 306 is drawn through the ligament and/or tissue 310. The fixation device 306 is drawn through the ligament/tissue until a desired length of fixation device is achieved for the proper suspension and/or tension.

In an embodiment, the fixation device 306 may be cut with a cutting instrument to remove any unwanted material. The fixation device 306 and the portion of the cut fixation device is retrieved external to the body through the exit port of the catch 122. In an embodiment, the delivery instrument 100 can be used for placement of additional fixation devices or needled sutures in the same patient.

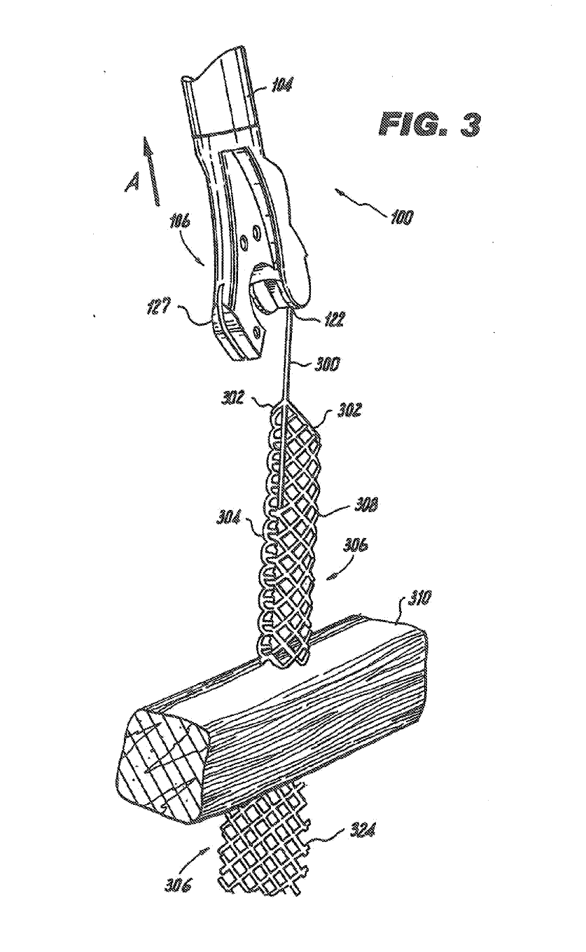

FIG. 4 shows a presently disclosed embodiment wherein a first fixation device 306a is engaged to a first end of a sling 324 and a second fixation device 306b is engaged to a second end of a sling 324. In an embodiment, a fixation device 306 is insert molded onto the sling material 324.

In an embodiment, the sling is an incontinence sling. Those skilled in the art will recognize that any type of sling is within the spirit and scope of the present invention.

In an embodiment, the presently disclosed delivery instrument 100 is used to place the sling ends through Cooper's ligament in a manner similarly described above and repeated on the contra lateral side. In an embodiment, the sling can be threaded in and out of the transobturators using a delivery instrument 100 via a single incision vaginal approach using a fixation device having a longer flexible shaft as shown in FIG. 4, for tensioning afterwards, through a mid-line incision.

In an embodiment as shown in FIG. 4, a plurality of barbs 312 are positioned along the shaft 300 of each fixation device 306a, 306b. The tissue and/or ligament may be anchored to by the barbs 312 positioned along the flexible shaft 300.

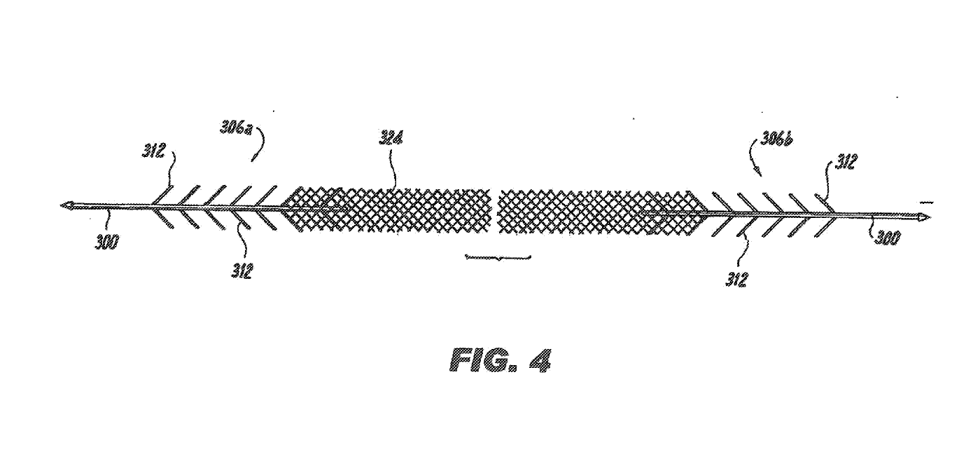

FIG. 5 shows a presently disclosed embodiment wherein a plurality of fixation devices 306a, 306b, 306c and 306d are engaged to a plurality of locations of a graft 324. In an embodiment, the fixation devices are 306a, 306b, 306c and 306d are insert molded onto a graft. Those skilled in the art will recognize that various processes may be used to engage a fixation device 306 to the graft and remain within the spirit and scope of the present invention.

The graft can be used for anterior or posterior repairs wherein the fixation devices 306a, 306b, 306c and 306d can be anchored to the sacrospinous ligament. Cardinal ligament, uterosacral ligament or other tissues and ligament to suspend the graft to support and repair prolapses and enterceles.

Each fixation device 306a, 306b, 306c and 306d is placed individually at the discretion of the user by drawing the fixation device 306a, 306b, 306c and 306d through ligament of tissue until the desired length of “leg” is achieved. As such, a head of the first fixation device 306a is engaged to the delivery instrument 100, driven through a tissue, secured in a catch, positioned in the tissue by withdrawing the delivery instrument 100, and disengaged from the catch. Next, the head of the second fixation device 306b is engaged to the delivery instrument 100 and the steps are repeated. The above-identified procedure is followed for all additional fixation devices 306c, 306d, etc.

In an embodiment, the graft is centered over the enterocele. In an embodiment, the graft is suspended non-taut.

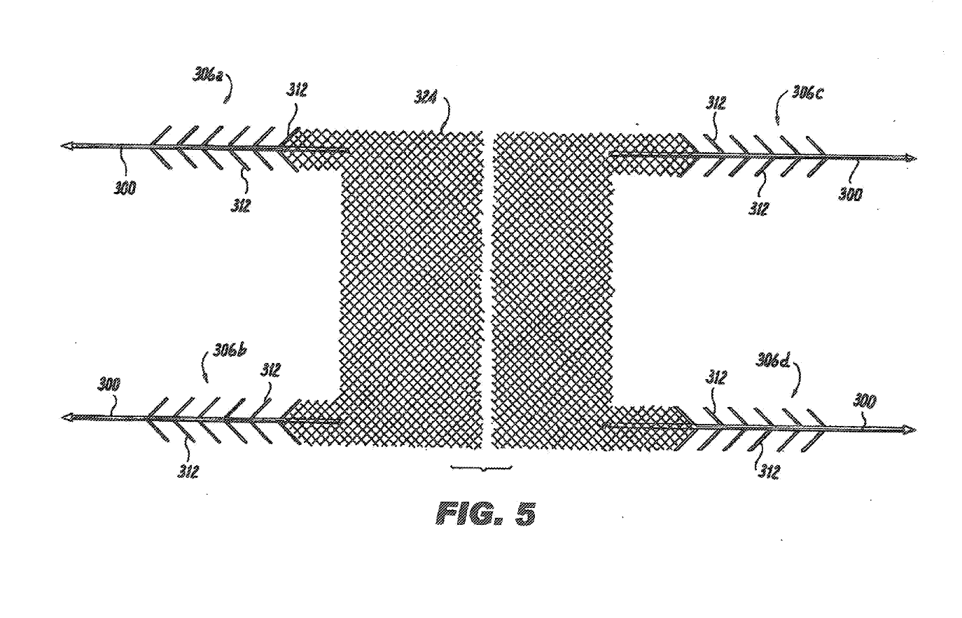

FIG. 6 shows a presently disclosed embodiment wherein a plurality of fixation devices 306a, 306b, 306c, and 306d are engaged to graft 324. In comparison to the fixation devices 306a, 306b, 306c and 306d shown in FIG. 5, the fixation devices 306a, 306b, 306c and 306d do not comprise a plurality of barbs 312; rather, the various shafts 300 comprise a plurality of protrusions 313 which allow the user to engage a desired protrusion 313 to the catch 122 to achieve a desired tension. As such, the position and the desired tension of the graft 324 is achieved by displacement of the draw of each fixation device 306a, 306b, 306c and 306d through a tissue and/or ligament. Additional draw can create tension in the graft 324.

The size and shape of the various components of a fixation device 306 and/or sling material 324 can vary for different applications. The shaft 300 length can vary from about 1 cm to about 120 cm. In an embodiment, the shaft 300 length is greater than about 120 cm. The longer shaft 300 length enables a second fixation device 306b to be withdrawn from the body after the first placement of the first fixation device 306a in order to be reloaded onto the carrier 124. The second fixation device 306b can also be placed into a different location to create a “suture bridge” for approximation or suspension.

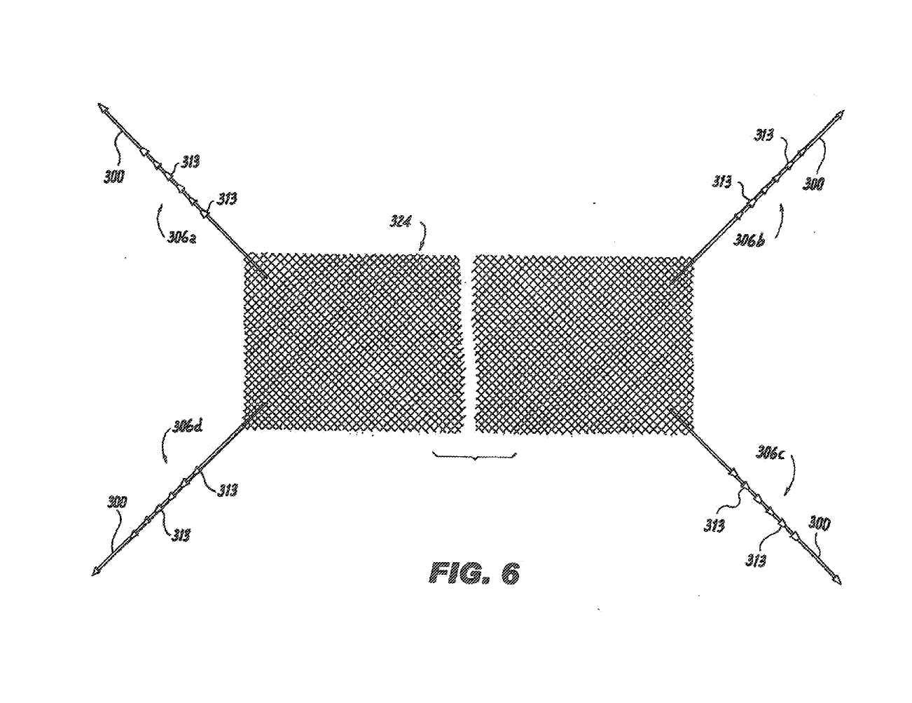

FIG. 7 shows a presently disclosed embodiment wherein the fixation device 306 is a separate unit. The fixation device head 328 is sized and shape fitted to the carrier 124 of the delivery instrument 100. The circular protrusions 313 allow tissue to pass in one direction and anchors in the opposing direction. A backstop 316 is provided to abut against tissue to prevent the fixation device 306 from passing through the tissue. In an embodiment, the backstop 316 is a circular shape. In an embodiment, the backstop 316 is a T-shaped protrusion. Those skilled in the art will recognize that any a backstop of any shape which prevents the fixation device from passing through the tissue is within the spirit and scope of the present invention.

FIG. 7 shows a presently disclosed embodiment wherein a fixation device 306 pierces a first tissue 320 and a second tissue 320 with a single “throw” of the delivery instrument 100. In an embodiment, the first tissue 320 is pierced by a first throw of the delivery instrument 100 and the second tissue 322 is pierced by a second throw of the delivery instrument 100. The head may be pulled in a direction represented by arrow “B” to force the second tissue 322 to approximate to the first tissue 320. In an embodiment, a rod or a stabilizer can be used in the direction of arrow “C” to approximate the first tissue 320 to the second tissue 322.

In an embodiment, the fixation device 306 is secured in place with a button (not shown) slid down over the shaft 300 to engage a desired barb 313.

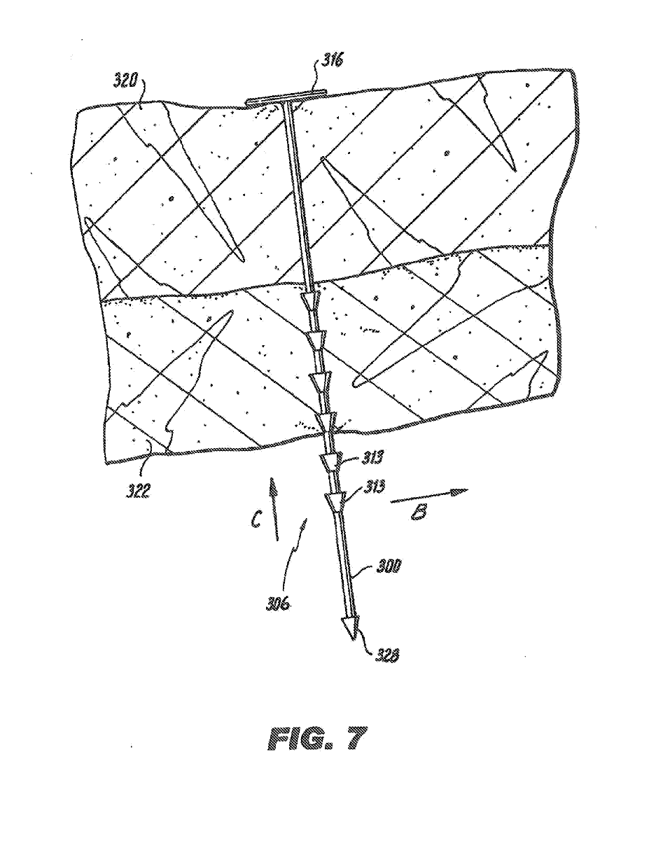

FIG. 8 shows a lock button 330 incorporated into an embodiment of the presently disclosed fixation device 306. In an embodiment, the lock button 330 is positioned to receive the carrier 124 and fixation device head 328. The lock button 330 has a lock diameter larger than the fixation device head 328 and carrier 124 such that the carrier 124 and fixation device head 328 pass through the lock button 330 freely but is sized to be received by the catch 124. To lock, the fixation device head 328 is pulled to draw the shaft 300 through the lock hole 330 to engage the locking barbs 313. Further drawing indexes to the next barb 313 tightening the resulting loop. Excess loop is trimmed.

Referring to FIG. 9A, in one embodiment, the fixation device head 328 includes a tip 130 and a shaft 134 coupled to the tip 130, thereby forming a shoulder 132. The shaft 134 is coupled to a shaft 300. The fixation device head 328 is inserted into the lumen 138 and held by a slight friction fit.

Referring again to FIGS. 1B and 1C, in operation, a user (such as a physician or other medical personnel) actuates the fixation device deployment mechanism 110 by pushing on the button 117, which via the attachment to the wireform 103 which is attached to the pusher wire 111, moves the coupling 150 along the track 152 concomitantly moving the carrier wire 154, which slidably moves the extendable carrier 124 through the exit port 120. The user continues to push the button 117 until the fixation device head 328 enters the catch 122. The catch 122, as shown in FIG. 9B, includes openings 170 defined by successive ribs 172. The catch 122 receives the fixation device head 328 (coupled to the shaft 300 of the fixation device 306) through opening 170, the ribs 172 deflect slightly to allow the fixation device head 328 to pass through. After the formed shoulder 132 has passed the ribs 172, the ribs 172 spring back to their original position defining the openings 170, and the fixation device head 328 remains captured in the catch 122. The user releases the button 117 and the spring 115 urges the button 117 proximally, moving the pusher wire 111, the coupling 150, the carrier wire 154, and the carrier 124 proximally along with the button 117 to the retracted position. As the extendable carrier 124 moves back to the retracted position, the fixation device head 328 slides out of the carrier. The openings 170 are chosen to be smaller in dimension than the formed shoulder 132. This causes the catch 122 to retain the fixation device head 328 because the flat rear surface of the shoulder 132 prevents the fixation device head 328 from passing back through the opening 170. When it is necessary to remove the fixation device head 328 from the catch 122, the fixation device head 328 may be moved toward an enlarged portion 174 of opening 172. The enlarged portion 174 is sized to allow the formed shoulder 132 to pass through without resistance. The catch 122 is preferably constructed of thin stainless steel of high temper, such as ANSI 301 full hard. The catch 122 may be fabricated by means of stamping, laser machining, or chemical etching. Those skilled in the art will recognize that the catch may be comprise a wide range of materials and remain within the spirit and scope of the present invention; further, those skilled in the art will recognize that the catch may be fabricated by a wide range of methods and remain within the spirit and scope of the present invention.

The delivery instrument's component materials should be biocompatible. For example, the handle 102, the elongate body member 104, and portions of the fixation device head deployment mechanism 110 may be fabricated from extruded, molded, or machined plastic material(s), such as polypropylene, polycarbonate, or glass-filled polycarbonate. Other components, for example the fixation device head 328, may be made of stainless steel. Other suitable materials will be apparent to those skilled in the art. Additionally, the mechanical components and operation are similar in nature to those disclosed in U.S. Pat. Nos. 5,364,408 and 6,048,351, each of which is incorporated by reference herein in its entirety.

Certain embodiments according to the invention have been disclosed. These embodiments are illustrative of, and not limiting on, the invention. Other embodiments, as well as various modifications and combinations of the disclosed embodiments, are possible and are within the scope of this disclosure. Various other embodiments of the present invention are contemplated as being within the scope of the filed claims particularly pointing out and distinctly claiming the subject matter regarded as the invention.

1. A kit comprising:
a tissue fixation device having a head, a main body, and at least one shoulder; and
a delivery instrument including a fixation device deployment mechanism having an extendable carrier for deploying the fixation device;
wherein the extendable carrier is extended to allow the fixation device head to pierce a tissue mass and be received in a catch.

2. The kit of claim 1, wherein the fixation device head is pointed to spread a slot of the catch as it passes into the catch and wherein the slot then narrows as the extendable carrier retracts, trapping the head by a wider portion of the shoulder within the catch.

3. The kit of claim 1, wherein the fixation device head passes through a tissue and is secured in the catch.

4. The kit of claim 1 wherein fixation device head is retained in the catch of the delivery instrument.

5. The kit of claim 1, wherein when the delivery instrument is pulled away from the tissue in a general direction of arrow “A”.

6. The kit of claim 1, wherein the delivery instrument is configured in such a way so that when withdrawn from the tissue, the fixation device is drawn through the tissue.

7. The kit of claim 1, wherein the tissue includes ligaments.

8. The kit of claim 1, wherein the fixation device is drawn through the tissue until a desired length of fixation device is achieved for proper suspension and/or tension.

9. The kit of claim 1, wherein the fixation device may be cut with a cutting instrument to remove any unwanted material.

10. The kit of claim 9, wherein a portion of the cut fixation device is retrieved external to a body containing the tissue through an exit port of the catch.

11. The kit of claim 1, wherein the delivery instrument is used for placement of needled sutures.

12. The kit of claim 1, wherein a first fixation device is engaged to a first end of a mesh and a second fixation device is engaged to a second end of a mesh.

13. The kit of claim 12, wherein delivery instrument is used to place mesh ends through Cooper's ligament in a manner similarly described above and repeated on a contra lateral side.

14. The kit of claim 12, wherein the mesh can be threaded in and out of the transobturators using the delivery instrument via a single incision vaginal approach; and the fixation device having a longer flexible shaft for tensioning afterwards, through a mid-line incision.

15. A kit for repairing comprising:
a graft;
a plurality of fixation devices engaged to the graft, each having a head, a main body, and at least one shoulder;
a delivery instrument including a fixation device deployment mechanism having an extendable carrier for deploying the fixation device; and
an extendable carrier that is extended to allow the fixation device head to pierce a tissue mass and received in a catch;
wherein a plurality of fixation devices are engaged to the graft at a plurality of locations.

16. The kit of claim 15, wherein the graft is used for anterior or posterior repairs.

17. The kit of claim 15, wherein the fixation devices include legs that are to be anchored to a sacrospinous ligament, Cardinal ligament, uterosacral ligament or other tissues and ligaments to suspend the graft to support and repair prolapses and enterceles.

18. The kit of claim 17, wherein each fixation device is placed individually at the discretion of a user by drawing the fixation device through a ligament or tissue until the desired length of leg is achieved.

19. A method of using a kit to repair prolapse, comprising the steps of:
a) providing;
a kit comprising:
a graft;
a plurality of fixation devices engaged to the graft, each having a head, a main body, and at least one shoulder;
a delivery instrument including a fixation device deployment mechanism having an extendable carrier tor deploying the fixation device; and
an extendable carrier that is extendable from the fixation device;
b) engaging the head of a first fixation device to the delivery instrument,
c) driving the head through a tissue;
d) securing a catch;
e) positioning the head in tissue by withdrawing the delivery instrument; and
f) disengaging from the catch.

20. The method of claim 19, further comprising the steps of:
providing a plurality of fixation devices;
engaging a head of a one of the plurality of fixation devices to the delivery instrument;
repeating steps c) through f) until all fixation devices suspend the graft to support and repair prolapse.
